(19) United States
(12) Patent Application Publication
(10) Pub. No.: US 2021/0327588 A1
(43) Pub. Date: Oct. 21, 2021

(54) METHODS, SYSTEMS, AND DEVICES FOR ASSESSING THE STATUS OF AN INDIVIDUAL'S IMMUNE SYSTEM

(71) Applicant: uMethod Health, Inc., Raleigh, NC (US)

(72) Inventors: John Quillian Walker, II, Mountain City, TN (US); Mark Clarence Zelek, Cary, NC (US)

(21) Appl. No.: 17/232,921

(22) Filed: Apr. 16, 2021

Related U.S. Application Data
(60) Provisional application No. 63/010,727, filed on Apr. 16, 2020.

(51) Int. Cl. G16H 50/30; G16H 15/00; G16H 20/10; G16H 10/60

(52) U.S. Cl. CPC G16H 50/30; G16H 10/60; G16H 20/10; G16H 15/00

(57) ABSTRACT

Methods, systems, and devices for solving the problem of more efficiently and consistently determining immune system status are disclosed herein. In one embodiment, a method includes receiving previous medical data associated with the first person, receiving current medical data associated with the first person, and determining immune system status data associated with the immune system based at least partially on the previous medical data and the current medical data. The method also includes generating a report including the immune system status data and transmitting the report to a graphical user interface (GUI), a printer, a computer log, and/or the like. The immune system status data may also include an overall assessment factor of the immune system of the first person.

### PRIORITY CLAIM

This application claims priority to U.S. Provisional Patent Application Ser. No. 63/010,727 filed Apr. 16, 2020, titled “METHODS, SYSTEMS, AND DEVICES FOR ASSESSING THE STATUS OF AN INDIVIDUAL'S IMMUNE SYSTEM” (Attorney Docket No. 567/8 PROV) the disclosure of which is incorporated herein by reference in its entirety.

### TECHNICAL FIELD

The present invention relates generally to medical diagnostics. More specifically, methods, systems, and devices are disclosed for assessing the status of the immune system for individuals.

### BACKGROUND

A key health trait for any individual is the overall performance of their immune system. The immune system is extremely important in protecting the individual from germs that include bacteria and viruses. The immune system can also detect harmful environmental substances and neutralize them. The immune system further protects for harmful cell changes (e.g., cancer cells, etc.). The immune system includes proteins, cells, and various organs. It is a necessity for health care providers to understand how well a given person's immune system is working. Determining the overall immune system status quickly and efficiently in emergency room settings and/or other triage environments is critical for saving lives and reducing risk of additional diseases and/or harm to those under their care. It can be used in doctor's offices as part of diagnosing how to respond to acute and chronic diseases, as well as for day-to-day visits. Individuals can assess the status of their own immune system, to understand their own health status, as a follow-on to routine blood draws.

As such, new methods, systems, and devices are needed to determine the overall immune system status for an individual efficiently.

### SUMMARY

Disclosed herein are methods, systems, and devices for solving the problem of more efficiently and consistently determining immune system status. Additionally, an immune system assessment score (i.e. an overall assessment factor of a person's immune system) is disclosed that is consistent, repeatable and predictable. This can provide guidance for a current situation, but it also provides a means of tracking changes over time, so that any degradations in the immune system can be detected and addressed before they become a problem; in contrast, improvements can be readily observed. The person can compare past and present assessment scores and also compare across people, including across whole populations. Such an assessment score allows for many types of statistical studies. According to one embodiment, a method is implemented on at least one computing device for assessing the immune system of a first person. The method includes receiving previous medical data associated with the first person, receiving current medical data associated with the first person, and determining immune system status data associated with the immune system based at least partially on the previous medical data and the current medical data. The method also includes generating a report including the immune system status data and transmitting the report to a graphical user interface (GUI), a printer, a computer log, and/or the like. The immune system status data may also include an overall assessment factor of the immune system of the first person.

In some embodiments, the previous medical data may include a plurality of previous medical values and the current medical data may include a plurality of current medical values. Determining the immune system status data may include normalizing the plurality of previous medical values to provide normalized previous medical values, and normalizing the plurality of current medical values to provide normalized current medical values. Determining the immune system status data may further include applying a plurality of previous data weights to the plurality of normalized previous medical values to provide weighted normalized previous medical values, and applying a plurality of current data weights to the plurality of normalized current medical values to provide weighted normalized current medical values. The overall assessment factor of the immune system may be further determined using the weighted normalized previous medical values and the weighted normalized current medical values. In certain embodiments, the method may include receiving the plurality of previous data weights over a data communication interface and receiving the plurality of current data weights over the data communication interface.

In some embodiments, the previous medical data may include blood test data. The blood test data may include a comprehensive metabolic panel (CMP) associated with the first person. The blood test data may also include a complete blood count (CBC). The previous medical data may also include an electronic health record (EHR), an electronic medical record (EMR), or the like. The current medical data may include a medication list, a problem list (i.e., list of active comorbidities), and genetic data of the first person. The current medical data may also include age, sex, height and weight of the first person. At least a portion of the previous medical data may be received via a data communication interface. The data communication interface may be a local area network (LAN) interface, a wide area network (WAN) interface, a personal area network (PAN), or the like. In certain embodiments, at least a portion of the previous medical data may be received via an electronic health record (EHR) application programing interface (API). In some embodiments, at least a portion of the previous medical data and or the current medical data may be received via the GUI, a camera, a scanner, an on-line facsimile (fax), a short message service (SMS) interface, a Multimedia Messaging Service (MMS) interface, an email account interface, and or the like.

In some embodiments, the first person immune system assessment report may include at least a portion of the previous medical data and the GUI may be configured to allow the portion of the previous medical data to be varied by a user. Upon varying the portion of the previous medical data via the GUI, the method may also include determining updated immune system status data and transmitting the updated immune system status data to the GUI.

In some embodiments, the first person immune system assessment report may include at least a portion of the current medical data and the GUI may be configured to allow the portion of the current medical data to be varied by a user. Upon varying the portion of the current medical data via the GUI, the method may also include determining updated immune system status data and transmitting the updated immune system status data to the GUI.

In some embodiments, the first person may be under care of a health care provider and the GUI may be configured for the health care provider. In other embodiments, the GUI may be configured for the first person.

In some embodiments, the method may be implemented at least partially within a tablet, smartphone, smartwatch, personal computer, or the like. The method may also be implemented at least partially within one or more servers.

In some embodiments, the method may be implemented at least partially within a fitness tracker. At least a portion of the current medical data may be provided by one or more sensors embedded within the fitness tracker.

According to another embodiment, a computing device includes a memory and at least one processor configured to provide a method for assessing an immune system of a first person. The method includes receiving previous medical data associated with the first person, receiving current medical data associated with the first person, and determining immune system status data associated with the immune system based at least partially on the previous medical data and the current medical data. The method also includes generating a report including the immune system status data and transmitting the report to a GUI, a printer, computer log, and/or the like. The immune system status data may also include an overall assessment factor of the immune system of the first person.

According to another embodiment, a non-transitory computer-readable storage medium stores instructions to be implemented on at least one computing device including at least one processor. The instructions when executed by the at least one processor cause the at least one computing device to provide a method for assessing an immune system of a first person. The method includes receiving previous medical data associated with the first person, receiving current medical data associated with the first person, and determining immune system status data associated with the immune system based at least partially on the previous medical data and the current medical data. The method also includes generating a report including the immune system status data and transmitting the report to a GUI, a printer, a computer log, and/or the like. The immune system status data may also include an overall assessment factor of the immune system of the first person.

According to another embodiment, a method is disclosed for assessing an immune system of a first person. The method includes questioning at least one of the first person or a responsible party of the person, by at least one health care provider. The health care provider obtains previous medical data and current medical data associated with the first person. The health care inputs to a user interface (UI) the previous medical data and the current medical data.

At least one computing device associated with the user UI, determines immune system status data associated with the immune system based on the previous medical data and the current medical data. Next, the computing device generates a first person immune system assessment report including the immune system status data. The computing device then transmits the person's immune system assessment report to a GUI, a printer, computer log, and/or the like. The immune system status data may also include an overall assessment factor of the immune system of the first person. In certain embodiments, the GUI is the UI.

In some embodiments, the health care worker determines a course of treatment for the first person based at least partially on the first person immune system assessment report. In certain embodiments, the health care worker may also determine a priority level versus a plurality of other people using a plurality of other person's immune system assessment reports.

According to another embodiment, a method is implemented on at least one computing device for assessing the immune system of a first person. The method includes receiving current medical data associated with the first person, and determining immune system status data associated with the immune system based at least partially on the current medical data. The method also includes generating a report including the immune system status data and transmitting the report to a graphical user interface (GUI), a printer, a computer log, and/or the like. The immune system status data may also include an overall assessment factor of the immune system of the first person.

In some embodiments, the current medical data may include a plurality of current medical values. Determining the immune system status data may include normalizing the plurality of current medical values to provide normalized current medical values. Determining the immune system status data may further include applying a plurality of current data weights to the plurality of normalized current medical values to provide weighted normalized current medical values. The overall assessment factor of the immune system may be further determined using the weighted normalized current medical values. In certain embodiments, the method may include receiving the plurality of current data weights over a data communication interface.

In some embodiments, the current medical data may include blood test data. The blood test data may include a comprehensive metabolic panel (CMP) associated with the first person. The blood test data may also include a complete blood count (CBC).

According to another embodiment, a computing device includes a memory and at least one processor configured to provide a method for assessing an immune system of a first person. The method includes receiving current medical data associated with the first person, and determining immune system status data associated with the immune system based at least partially on the current medical data. The method also includes generating a report including the immune system status data and transmitting the report to a graphical user interface (GUI), a printer, a computer log, and/or the like. The immune system status data may also include an overall assessment factor of the immune system of the first person.

According to another embodiment, a non-transitory computer-readable storage medium stores instructions to be implemented on at least one computing device including at least one processor. The instructions when executed by the at least one processor cause the at least one computing device to provide a method for assessing an immune system of a first person. The method includes receiving current medical data associated with the first person, and determining immune system status data associated with the immune system based at least partially on the current medical data. The method also includes generating a report including the immune system status data and transmitting the report to a graphical user interface (GUI), a printer, a computer log, and/or the like. The immune system status data may also include an overall assessment factor of the immune system of the first person.

According to another embodiment, a method is disclosed for assessing an immune system of a first person. The method includes questioning at least one of the first person or a responsible party of the person, by at least one health care provider. The health care provider obtains current medical data associated with the first person. The health care then inputs to a user interface (UI) the current medical data.

At least one computing device associated with the user UI, determines immune system status data associated with the immune system based on the current medical data. Next, the computing device generates a first person immune system assessment report including the immune system status data. The computing device then transmits the person's immune system assessment report to a GUI, a printer, a computer log, and/or the like. The immune system status data may also include an overall assessment factor of the immune system of the first person. In certain embodiments, the GUI is the UI.

In some embodiments, the health care worker determines a course of treatment for the first person based at least partially on the first person immune system assessment report. In certain embodiments, the health care worker may also determine a priority level versus a plurality of other people using a plurality of other person's immune system assessment reports.

The features and advantages described in this summary and the following detailed description are not all-inclusive. Many additional features and advantages will be apparent to one of ordinary skill in the art in view of the drawings, specification, and claims presented herein.

### BRIEF DESCRIPTION OF THE DRAWINGS

The present embodiments are illustrated by way of example and are not intended to be limited by the figures of the accompanying drawings. In the drawings:

FIG. 1 depicts a flow chart illustrating a method including steps for assessing an immune system of a first person in accordance with embodiments of the present disclosure.

FIG. 2A and FIG. 2B depict page 1 and page 2 of an example of an immune system assessment report for an individual in accordance with embodiments of the present disclosure.

FIG. 3 depicts a system including a hospital server and a tablet for assessing an immune system of an individual in accordance with embodiments of the present disclosure.

FIG. 4 depicts a block diagram illustrating the tablet device of FIG. 3 for assessing an immune system of an individual in accordance with embodiments of the present disclosure.

FIG. 5 depicts a block diagram illustrating the hospital server of FIG. 3 for assessing an immune system of an individual in accordance with embodiments of the present disclosure.

FIG. 6 depicts a block diagram illustrating a personal computer (PC) that may be used in place of the tablet device of FIG. 3 for assessing an immune system of an individual in accordance with embodiments of the present disclosure.

### DETAILED DESCRIPTION

The following description and drawings are illustrative and are not to be construed as limiting. Numerous specific details are described to provide a thorough understanding of the disclosure. However, in certain instances, well-known or conventional details are not described in order to avoid obscuring the description. References to “one embodiment” or “an embodiment” in the present disclosure can be, but not necessarily are, references to the same embodiment and such references mean at least one of the embodiments.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way.

Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and is not intended to further limit the scope and meaning of the disclosure or of any exemplified term. Likewise, the disclosure is not limited to various embodiments given in this specification.

Without intent to limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

Disclosed herein are methods, systems, and devices for solving the problem of more efficiently and more consistently determining immune system status.

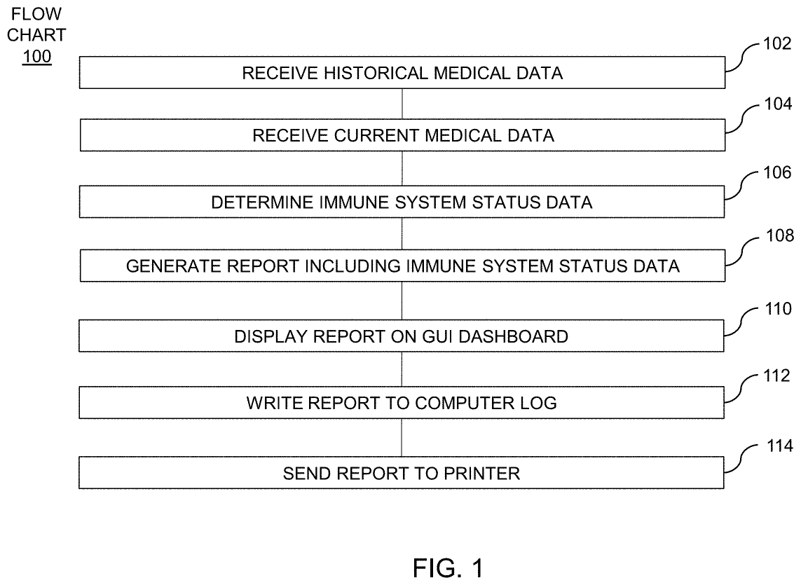

FIG. 1 depicts a flow chart 100 illustrating a method including steps for assessing an immune system of a first person in accordance with embodiments of the present disclosure. The method may be executed on one or more computer devices having one or more processors. The method may be executed on a client device such as a tablet, smartphone, fitness device, smartwatch, personal computer, or the like. The method may also be executed on server and connected to a client device over a network. The server may be housed within a business arrangement or may be housed within a data center. The method may be executed partially on a server and partially on a client device.

In step 102, the one or more computer devices receive previous medical data associated with an individual. The previous medical data may include blood test data that also includes a comprehensive metabolic panel (CMP). The blood test data may also include a complete blood count (CBC). The previous medical data may further include an electronic health record (EHR), an electronic medical record (EMR), or the like. The previous medical data may be received over a network or may be entered via a graphical user interface (GUI) associated with the client device. The network may include a local area network, a wide area network (WAN) interface, and/or a personal area network (PAN). The GUI may be provided by a tablet, smartphone, a smartwatch, a laptop, a personal fitness device, a personal computer, or the like. The GUI may be further provided by a web browser such as a Microsoft Internet Explorer® browser, a Microsoft Edge® browser, an Apple Safari® browser, a Google Chrome® browser, a Mozilla Firefox® browser, an Opera® browser, or the like. In other embodiments, the GUI may be further provided by a dedicated application developed specifically for assessing an immune system of a person.

In step 104, the one or more computer devices receive current medical data associated with the individual. The current medical data may include a medication list of the first person. The current medical data may also include age, weight, gender, body mass index (BMI), lifestyle, diet, drinking, smoking, or the like. The current medical data may be received via the GUI. At least a portion of the current medical data may also be received over the network or via another GUI, a camera, a scanner, an on-line facsimile (fax), a short message service (SMS) interface, a Multimedia Messaging Service (MMS) interface, an email account interface, and/or the like.

In step 104, the one or more computer devices receive current medical data associated with the individual. The current medical data may include a problem list of the first person. This problem list identifies conditions and comorbidities, when they were first diagnosed, whether they are currently active, and whether they are being currently treated. The current problem list data may be received via the GUI. At least a portion of the current problem list data may also be received over the network, or via another GUI, a camera, a scanner, an on-line fax, an SMS interface, an MMS interface, an email account interface, and/or the like.

In step 104, the one or more computer devices receive current medical data associated with the individual. The current medical data may include genetic data of the first person. This genetic data may be a whole genome or whole exome analysis, or it may be a partial analysis (such as provided by 23andMe in its raw data file). The current genetic data may be received via the GUI or other suitable input medium. At least a portion of the current genetic data may also be received over the network.

In step 106, the one or more computer devices determine immune system status data including an overall assessment factor of the immune system based at least partially on the previous medical data and the current medical data. More specifically, assessing the immune system status includes normalizing the plurality of previous medical values to provide normalized previous medical values, and normalizing the plurality of current medical values to provide normalized current medical values. Determining the immune system status data then includes applying a plurality of previous data weights to the plurality of normalized previous medical values to provide weighted normalized previous medical values, and applying a plurality of current data weights to the plurality of normalized current medical values to provide weighted normalized current medical values. The overall assessment factor of the immune system is then determined using the weighted normalized previous medical values and the weighted normalized current medical values. The plurality of previous data and the plurality of current data weights may be received and updated over the data communication interface.

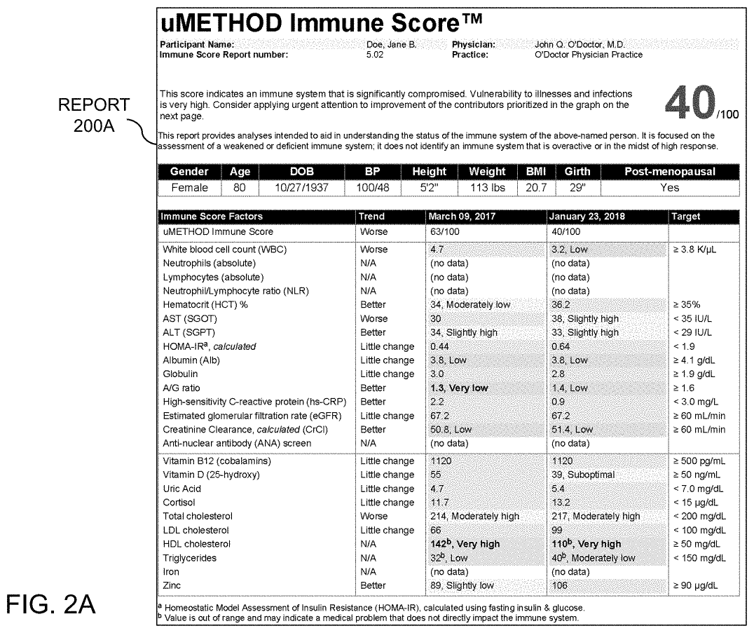

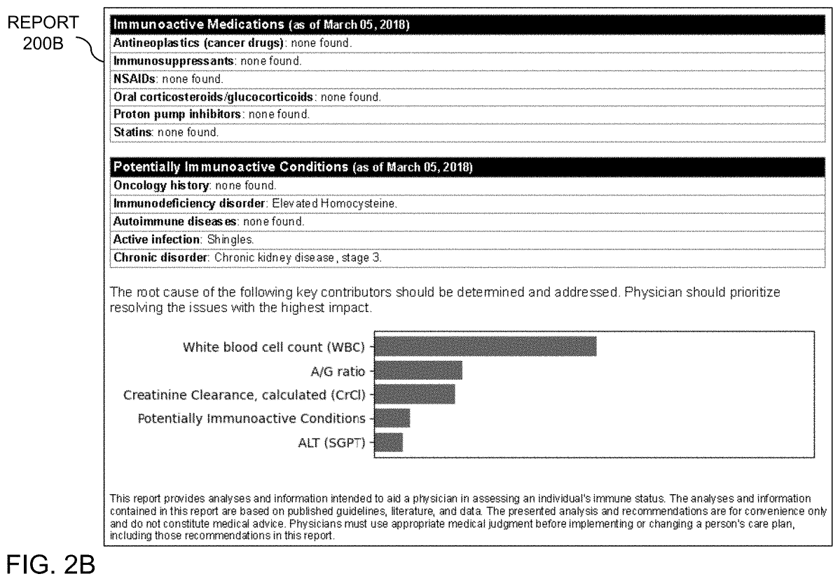

In step 108, the one or more computer devices generate an immune system assessment report including the immune system status data associated with the individual. An example of an immune system assessment report is disclosed later in FIG. 2A and FIG. 2B.

In step 110, the one or more computer devices transmit all or at least a portion of the immune system assessment report to the GUI.

In step 112, the one or more computer devices write all or at least a portion of the immune system assessment report to a computer log.

In step 112, the one or more computer devices transmit all or at least a portion of the immune system assessment report to a printer.

FIG. 2A and FIG. 2B depict page 1 (report 200A) and page 2 (report 200B) of an example of an immune system assessment report for an individual in accordance with embodiments of the present disclosure. The immune system assessment report includes an overall assessment factor of 40 out of a possible 100 rating for the immune system of the individual. The goal of the immune system assessment report is to give a single number on a one-page report that gives a medical professional or the person themselves a quantifiable assessment of the status of their immune system. Specifically, to identify if the person is considered to be “immuno-compromised” or to have a weakened immune system, is trending in that direction, or may be considered to have an immune system that would be characterized as healthy.

An 80 or greater overall assessment factor could indicate a strong immune system of the individual. Basically, no known immune compromising drivers have excessively declined. Healthy, active, good diet might observe this assessment score. Most people in their teens, 20s, and 30s would typically be assessed with a score in this range. Such individuals most likely have good genetics and are taking good care of themselves. They probably go to the gym, eat organic food, and are high-functioning individuals.

A 50 to 80 overall assessment factor may be more representative of the average adult in Western culture. They may have few active medical issues identified in their problem list, but probably have a sedentary lifestyle, may be overweight, and may be stressed. Such a person may have the potential to improve their health (including the status of their immune system) or they may let it decline further. Probably 60% of the population in their 30s and 40s may fall in this range. Typically, a doctor and/or health care provider may not take urgent action for the person falling in this scoring range.

A 30 to 50 overall assessment factor could be considered below average. This assessment range may be due to emerging or current medical issues, including some that may be exacerbated by age. Such a person could be considered at increased risk of an increased inflammatory state or infections. Typically, a doctor or health care provider would recommend changes and/or take treatment actions for the individual.

A 0 to 30 overall assessment factor may indicate a compromised immune system for the individual. Older adults, where medical issues are advanced, may score here. Some individuals in their 30s and 40s may score here, based on current illnesses. Medications and medical issues are likely contributing to the decline of their immune system. HIV patients may be in this assessment range.

The example report contains the person's name (i.e., participant name), a report number, a physician name, and the physician's practice. One or more of these items may be omitted to protect the privacy of the individual. In some embodiments, the report may also contain a unique patient identifier (not shown). As a reference point, the example report depicts a previous overall assessment factor of 63 out of a possible 100 rating with a trend towards worse. The example report also depicts current and previous blood test data representative of a CBC and CMP for the individual. Trends and targets are also depicted for the serum blood tests. Additional information in the report includes that no immunoactive medications or autoimmune diseases were reported for the individual.

The overall assessment factor (i.e., uMethod Immune Score™) may be used in a triage scenario to determine those people that may be the most vulnerable. Facilities like nursing homes, assisted living, and memory care units may also benefit by better understanding those who may be more susceptible to infections and disease. In fact, any person and/or those caring for others may benefit.

In certain embodiments, the previous medical data and the current medical data may be segmented within the following five major input categories:

Biospecimen reports (e.g., blood and urine chemistry)

Active disease states

Medication list

Genetic data

Vitals and history

Blood and urine chemistry may be taken from recent lab work (e.g., less than 6 months old). If the lab work is not recent, the information may be omitted in the analysis or a request for more recent lab work may be requested. The results may be weighted appropriately based on contribution to the overall assessment factor. The following is a sample of results (but not limited to) that may be analyzed:

WBC count (low)

Neutrophils % (low)

Lymphocytes % (high)

Albumin (low), Globulin (low)

Hematocrit (low)

ESR (sed rate) (high)

ALT (high), AST (high)

HOMA-IR

Reference Lab alarm/critical conditions

High inflammatory state

Hepatic or renal insufficiency

Metal toxicity

The active disease states may be received as a problem list, be observed by the software engine, and/or observed by the health care professional. Active disease states may be weighted based on importance of each disease and its severity. For example, cancer may be weighted heavily. Overall examples may include (but not limited to) the following:

Autoimmune diseases

Immunodeficiency disorders (such as HIV)

Chronic diseases

Cancer & cancer therapy, depression, leaky gut syndrome

Examples of the medication list that may be weighted heavily include (but not limited to) the following:
Immunosuppressants, oral steroids, excessive opioids/opiates
Drugs that indicate an active chronic disease not otherwise reported or observed
Too many drug interactions (DDIs)
The presence of critical DDIs (those interactions to be avoided, known as type X)
Drugs that can exacerbate the conditions, including, but not limited to, mentioned in the previous paragraph
Drug-drug interactions that can increase the risk or severity of the conditions, including, but not limited to, mentioned in the previous paragraph
   Specific drugs may indicate specific diseases as well. For example, certain drugs may indicate uncontrolled type-2 diabetes. That is, basic therapeutics have probably been applied; stronger therapeutics may indicate the continued presence of the disease, and that earlier treatments have not had the desired outcomes.

Vitals and personal history may include (but not limited to) the following:

Age and gender

Obesity (as determined by factors such as weight and height)

Transplants, recent surgeries, accidents or other incidents

Smoking status, vaping status, excessive alcohol consumption

In some embodiments, the immune system assessment report may flag stale data and or missing data that may affect the quality and/or confidence in the overall assessment factor. The immune system assessment report may be configured as either a single page report for the printer or a two-page report printed on a single sheet. The immune system assessment report may also depict a score interpretation of the overall assessment factor. Recommendations for improvement may also be depicted on the immune system assessment report.

In some embodiments, the methods presented herein may incorporate new or known artificial intelligence (AI) techniques and algorithms for training/updating weights, and updating specific previous medical data and/or current medical data used.

In some embodiments, the methods previously discussed may be implemented without obtaining a current blood sample or urine sample or genetic data from the individual. Additionally family information and/or person history may not be needed. In certain embodiments, past surgeries and/or accidents may not be needed.

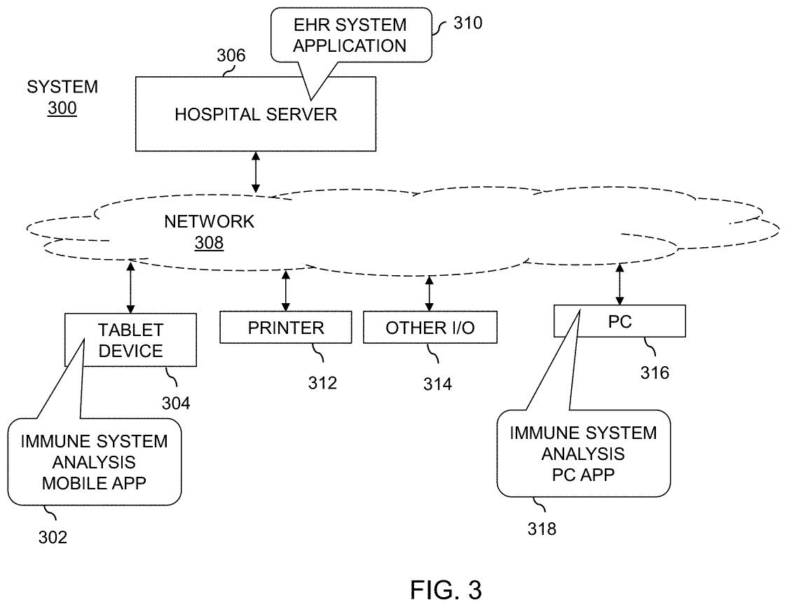

FIG. 3 depicts a system 300 for assessing an immune system of an individual in accordance with embodiments of the present disclosure. The system 300 includes an immune system analysis mobile application (app) 302 configured to be executed on a computer or tablet device 304. In certain embodiments, the immune system analysis mobile app may be a web browser as described with the flow chart 100 of FIG. 1. Input data can also be received by any media, such as fax, scanned sheets of paper, word-processor document files, or spreadsheets files. The table 304 includes a GUI configured for a health care provider. The tablet 304 is configured to communicate with a hospital server 306 over a network 308. The hospital server 306 is configured to execute an EHR system application 310. The flow chart 100 of FIG. 1 may be executed partially on the tablet 304 and partially on the server 306. Additionally, the method may be executed fully on the tablet 304 by the immune system analysis mobile app 302. Other input/output (I/O) 314 to the system 300 may include (but not limited to) another GUI, a camera, a scanner, an on-line fax, an SMS interface, an MMS interface, an email account interface, and/or the like. The other I/O 314 may be configured to receive multiple types of file formats such as Adobe®, Microsoft® Office, text, picture formats, raw data, or the like. The other GUI may be associated with one or more tablets, smartphones, smartwatches, laptops, personal computers, or the like. The system 300 also includes a printer 312 for receiving and printing the immune system assessment report. Not shown in system 300 is the computer log of flowchart 100. The computer log may be implemented as a database within the tablet device 304 and/or the hospital server 306, or the like.

The hospital server 306 may be implemented within a hospital or other localized entity. The hospital server 306 may also be implemented within a cloud computing environment such as the Microsoft Azure®, the Amazon Web Services® (AWS), or the like cloud computing data center environments. The hospital server 306 may be implemented as a virtual server such as an Ubuntu® server. The hospital server 306 may also be configured to be hosted within a virtual container. For example, the virtual container may be the Docker® virtual container or the like. In some embodiments, the virtual server and/or virtual container may be distributed over a plurality of hardware servers using hypervisor technology.

The system may be further configured to include a personal computer (PC) 316 running an immune system analysis PC application 318 coupled with the network 308 and in communication with the hospital server 306. The PC 316 and the immune system analysis PC application 318 may be configured to provide similar functionality to the tablet device 304 running the immune system analysis mobile app 302. In certain embodiments, the immune system analysis PC application may be a web browser as described with the flow chart 100 of FIG. 1. The PC 316 may also be configured as an administrative user interface (UI) for the system 300.

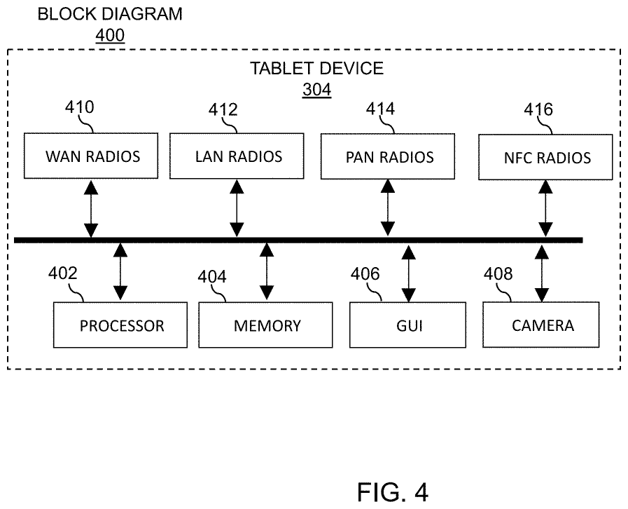

FIG. 4 depicts a block diagram 400 illustrating the tablet device 304 of FIG. 3 for assessing an immune system of an individual in accordance with embodiments of the present disclosure. The tablet device 304 includes a processor 402, a memory 404, and a GUI 406.

The processor 402 may be a mobile processor such as the Qualcomm® Snapdragon™ mobile processor. The memory 404 may include a combination of volatile memory (e.g., random access memory) and non-volatile memory (e.g., flash memory). The memory 404 may be partially integrated with the processor 402. The GUI 406 may be a touchpad display.

A camera 408 is included and may be used by the immune system analysis mobile app 302 of FIG. 3 to capture a quick response (QR) code and/or a barcode on a patient's hospital bracelet. The camera 408 may also be used to capture a portion of the previous medical data and/or current medical data. The tablet device 304 also includes WAN radios 410, LAN radios 412, and PAN radios 414 configured to communicate over the network 308 of FIG. 3. The WAN radios 410 may include 2G, 3G, 4G, and/or 5G technologies. The LAN radios 412 may include Wi-Fi technologies such as 802.11a, 802.11b/g/n, 802.11ac, and/or 802.11ax circuitry. The PAN radios 414 may include Bluetooth® technologies. Near field communication (NFC) radios 416 are included with the tablet 304 as well and may be used to capture personal identification information from the patient's hospital bracelet.

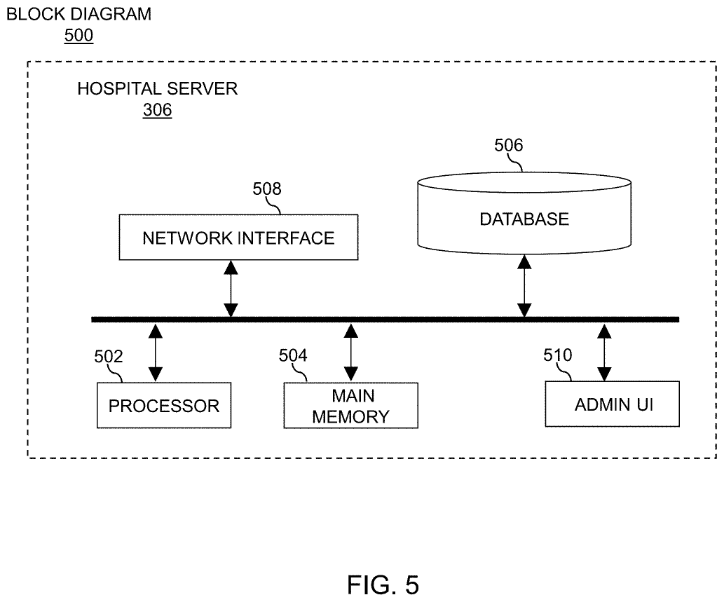

FIG. 5 depicts a block diagram 500 illustrating the hospital server 306 hosting the EHR system application 310 of FIG. 3 for assessing an immune system of an individual in accordance with embodiments of the present disclosure. The hospital server 306 may include at least one of a processor 502, a main memory 504, a database 506, a network interface 508, and an administration UI 510.

The processor 502 may be a multi-core server class processor suitable for hardware virtualization. The processor may support at least a 64-bit architecture and a single instruction multiple data (SIMD) instruction set. The main memory 504 may include a combination of volatile memory (e.g. random access memory) and non-volatile memory (e.g. flash memory). The database 506 may include one or more hard drives. The database 506 may provide long-term storage of immune system data recorded during assessments.

The enterprise network interface 508 may provide one or more high-speed communication ports to enterprise switches, routers, and/or network storage appliances. The enterprise network interface 508 may include high-speed optical Ethernet, InfiniBand (IB), Internet Small Computer System Interface iSCSI, and/or Fibre Channel interfaces. The administration UI may support local and/or remote configuration of the server 600 by a network administrator. The hospital server 306 hosting the EHR system application 310 and configured within system 300 is transformed to provide at least a portion of a machine that solves the problem of better determining an overall immune system status for an individual efficiently.

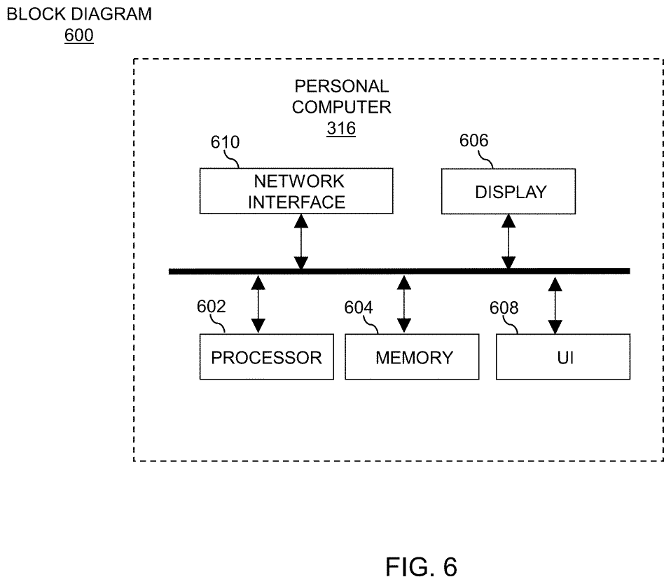

FIG. 6 depicts a block diagram 600 illustrating the PC 316 of FIG. 3 for assessing an immune system of an individual in accordance with embodiments of the present disclosure. The PC 316 may be configured to provide similar functionality to the tablet device 304 and/or as an administrative UI for the system 300 as previously described. The personal computer 316 may include at least a processor 602, at least one memory 604, a display 606, a UI 608, and a network interface 610. The personal computer 700 may include an operating system such as a Windows® OS, a Macintosh® OS, a Linux® OS, or the like. The memory 604 may include a combination of volatile memory (e.g. random access memory) and non-volatile memory (e.g. solid state drive and/or hard drives). The display 606 may be an external display (e.g. computer monitor) or internal display (e.g. laptop). The UI 608 may include a keyboard, and a pointing device (e.g. mouse). The network interface 610 may be a wired Ethernet interface or a Wi-Fi interface. The PC 316 may be configured to access the server 600 of FIG.6 and/or remote memory (e.g. network storage and/or cloud storage) via the network interface 712. The PC 316, when configured within system 300, is transformed to provide at least a portion of a machine that solves the problem of better determining an overall immune system status for an individual efficiently.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium (including, but not limited to, non-transitory computer readable storage media). A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including object oriented and/or procedural programming languages. For example, programming languages may include, but are not limited to: Ruby, JavaScript, Java, Python, Ruby, PHP, C, C++, C#, Objective-C, Go, Scala, Swift, Kotlin, OCaml, or the like.

Aspects of the present invention are described above with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions.

These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method implemented on at least one computing device for assessing an immune system of a first person; the method comprising:
receiving previous medical data associated with the first person
receiving current medical data associated with the first person;
determining immune system status data associated with the immune system based on the previous medical data and the current medical data;
generating a first person immune system assessment report including the immune system status data; and
transmitting the first person immune system assessment report to at least one of a graphical user interface (GUI), a printer, and a computer log.

2. The method of claim 1, wherein the immune system status data includes an overall assessment factor of the immune system of the first person.

3. The method of claim 2, wherein:
the previous medical data includes a plurality of previous medical values;
the current medical data includes a plurality of current medical values; and
determining the immune system status data includes:
normalizing the plurality of previous medical values to provide normalized previous medical values; and
normalizing the plurality of current medical values to provide normalized current medical values.

4. The method of claim 3, wherein determining the immune system status data further includes:
applying a plurality of previous data weights to the plurality of normalized previous medical values to provide weighted normalized previous medical values; and
applying a plurality of current data weights to the plurality of normalized current medical values to provide weighted normalized current medical values.

5. The method of claim 4 further comprising:
receiving the plurality of current data weights over a data communication interface; and
receiving the plurality of current data weights over the data communication interface.

6. The method of claim 4, wherein the overall assessment factor of the immune system is determined using the weighted normalized previous medical values and the weighted normalized current medical values.

7. The method of claim 1, wherein the previous medical data includes blood test data.

8. The method of claim 7, wherein the blood test data includes at least one of a complete blood count (CBC) and a comprehensive metabolic panel (CMP) associated with the first person.

9. The method of claim 1, wherein the previous medical data includes at least one of an electronic health record (EHR) and an electronic medical record (EMR).

10. The method of claim 1, wherein the current medical data includes a medication list of the first person.

11. The method of claim 1, wherein the current medical data includes a problem list of the first person.

12. The method of claim 1, wherein the current medical data includes genetic data of the first person.

13. The method of claim 1, wherein the current medical data includes vitals of the first person.

14. The method of claim 1, wherein at least a portion of the previous medical data is received via a data communication interface and the data communication interface is at least one of a local area network (LAN) interface a wide area network (WAN) interface, and a personal area network (PAN).

15. The method of claim 1, wherein at least a portion of the previous medical data is received via an electronic health record (EHR) application programing interface (API).

16. The method of claim 1, wherein at least a portion of the previous medical data is received via at least one of the GUI, a camera, a scanner, an on-line facsimile (fax), a short message service (SMS) interface, a Multimedia Messaging Service (MMS) interface, and an email account interface.

17. The method of claim 1, wherein at least a portion of the current medical data is received via the GUI, a camera, a scanner, an on-line facsimile (fax), a short message service (SMS) interface, a Multimedia Messaging Service (MMS) interface, and an email account interface.

18. The method of claim 1, wherein first person immune system assessment report includes at least a portion of the previous medical data.

19. The method of claim 18, wherein:
the GUI is configured to allow the portion of the previous medical data to be varied by a user; and
upon varying the portion of the previous medical data via the GUI, the method further comprises:
determining updated immune system status data; and
transmitting the updated immune system status data to the GUI.

20. The method of claim 1, where first person immune system assessment report includes at least a portion of the current medical data.

21. The method of claim 20, wherein:
the GUI is configured to allow the portion of the current medical data to be varied by a user; and
upon varying the portion of the current medical data via the GUI, the method further comprises:
determining updated immune system status data; and
transmitting the updated immune system status data to the GUI.

22. The method of claim 1, wherein the first person in under care of a health care provider and the GUI is configured for the health care provider.

23. The method of claim 1, wherein the GUI is configured for the first person.

24. The method of claim 1, wherein method is implemented at least partially within at least one of a tablet, a smartphone, a smartwatch, a laptop, and a personal computer.

25. The method of claim 1, wherein method is implemented at least partially within one or more servers.

26. The method of claim 1, wherein method is implemented at least partially within a fitness tracker.

27. The method of claim 26, wherein at least a portion of the current medical data is provided by one or more sensors embedded within the fitness tracker.

28. A computing device for assessing an immune system of a first person; the computing device comprising:
a memory; and
at least one processor configured for:
receiving previous medical data associated with the first person
receiving current medical data associated with the first person;
determining immune system status data associated with the immune system based on the previous medical data and the current medical data;
generating a first person immune system assessment report including the immune system status data; and
transmitting the first person immune system assessment report to at least one of a graphical user interface (GUI), a printer, and a computer log.

29. A non-transitory computer-readable storage medium, the non-transitory computer-readable storage medium storing instructions to be implemented on at least one computing device including at least one processor, the instructions when executed by the at least one processor cause the at least one computing device to perform a method for assessing an immune system of a first person; the method comprising:
receiving previous medical data associated with the first person
receiving current medical data associated with the first person;
determining immune system status data associated with the immune system based on the previous medical data and the current medical data;
generating a first person immune system assessment report including the immune system status data; and
transmitting the first person immune system assessment report to at least one of a graphical user interface (GUI), a printer, and a computer log.

30. A method for assessing an immune system of a first person; the method comprising:
questioning at least one of the first person and a responsible party, by at least one health care provider, to obtain previous medical data and current medical data associated with the first person.
inputting to a user interface (UI), by the at least one health care provider, the previous medical data and the current medical data;
determining, by at least one computing device associated with the user interface, immune system status data associated with the immune system based on the previous medical data and the current medical data;
generating, by the at least one computing device, a first person immune system assessment report including the immune system status data; and
transmitting, by at least one computing device, the first person immune system assessment report to at least one of a graphical user interface (GUI), a printer, and a computer log.
